(19) United States
(12) Patent Application Publication
(10) Pub. No.: US 2024/0241139 A1
(43) Pub. Date: Jul. 18, 2024

(54) DIAGNOSIS OF AUTISM SPECTRUM DISORDER BY MULTIOMICS PLATFORM

(71) Applicant: YISSUM RESEARCH DEVELOPMENT COMPANY OF THE HEBREW UNIVERSITY OF JERUSALEM LTD., Jerusalem (IL)

(72) Inventor: Haitham AMAL, Haifa (IL)

(73) Assignee: YISSUM RESEARCH DEVELOPMENT COMPANY OF THE HEBREW UNIVERSITY OF JERUSALEM LTD., Jerusalem (IL)

(21) Appl. No.: 18/563,900

(22) PCT Filed: May 25, 2022
(86) PCT No.: PCT/IL2022/050555

§ 371 Date: Nov. 23, 2023

Related U.S. Application Data
(60) Provisional application No. 63/192,638, filed on May 25, 2021.

(51) Int. Cl. G01N 33/68; G16H 50/20

(52) U.S. Cl. CPC G01N 33/6896; G16H 50/20; G01N 2440/14; G01N 2440/26; G01N 2800/28

(57) ABSTRACT

The present invention is directed to methods for determining an autism spectrum condition in a subject. Further provided is a kit suitable for determining an autism spectrum condition.

### CROSS-REFERENCE TO RELATED APPLICATIONS

This application claims the benefit of priority of U.S. Provisional Patent Application No. 63/192,638, titled “DIAGNOSIS OF AUTISM SPECTRUM DISORDER BY MULTIOMICS PLATFORM”, filed May 25, 2021, the contents of which are incorporated herein by reference in their entirety.

### FIELD OF INVENTION

The present invention is in the field of diagnosis of autism spectrum disorder.

### BACKGROUND OF THE INVENTION

Autism spectrum disorder (ASD) is a heterogeneous neurodevelopmental disorder caused by genetic modifications as well as non-genetic factors, associated with social communication deficits, repetitive behaviors, and restricted interest. About 1.8% of children have been identified with ASD according to CDC's Autism and Developmental Disabilities Monitoring (ADDM) Network estimations. Although Autism had been investigated since 1943 there is still no specific biomarker for diagnoses, and it is based mainly on criteria that were set by the American Psychiatric Association in the fifth edition of its Diagnostic and Statistical Manual of Mental Disorders (DSM-5).

Common molecular mechanisms, converging onto similar behavioral deficits, may exist in ASD patients. These mechanisms may be utilized to determine ASD biomarkers as well as molecular targets for ASD treatment.

Blood may be considered a first source for biomarkers. Changes in ASD biomarkers are manifested in protein levels, enzyme activity, and different post-translational modifications (PTMs). Thus, different clinical studies have shown changes in expression and activity of the proteins related to the inflammation and immune systems, proteins related to lipid and cholesterol metabolism, oxidative stress, and defective mitochondrial energy production in the blood of ASD patients. It should be noted that the loss of the blood-brain barrier integrity is common to patients with neurodevelopmental disorders, such as ASD, and neurodegenerative diseases. Hence, the molecules produced or modified in the brain may leak into the systemic circulation. These data imply that molecular alterations observed in the blood of patients with brain disorders reflect, at least in part, the changes taking place in the brain of these individuals. The following combination of three types of proteomics will be conducted to reveal the pathological molecular alterations observed in the plasma:

1) Global Proteomics is a powerful tool to provide large-scale analyses of protein expression in cells or tissues and enable the evaluation of proteins that are differentially expressed in different groups and can determine pathways and protein-protein interaction networks that are relevant to autism biology.

2) S-nitrosylation (SNO): SNO is a PTM and is caused by the reaction of nitric oxide (NO) with the sulfhydryl groups of the amino acid cysteine in the proteins resulting in the formation of S-nitrosothiols. Protein SNO regulates the localization and activity of many key enzymes and receptors. In physiological conditions, it modulates various biological processes in the brain, including synaptic plasticity, axonal elongation, and neuronal survival. However, aberrant SNO can cause protein misfolding, synaptic damage, mitochondrial fission, or apoptosis. The inventor and others have found that SNO can play an important role in the pathogenesis of different kinds of neurodegenerative disorders, such as Alzheimer's, Parkinson's, Huntington's, and other neurological diseases. The present inventor recently found that the Shank3 mutation in mice, representing one of the most promising models of ASD, leads to reprogramming of the SNO-proteome and established that NO may play a key role in the Shank3 pathology. These results agree with previous postmortem examinations of ASD patients showing the accumulation of 3-nitrotyrosine (3-Ntyr) in the brain due to autism. 3-Ntyr is generated by the interaction of peroxynitrite with tyrosine residues in the presence of elevated NO levels and represents a marker of oxidative/nitrosative stress, DNA damage, and cell death. Importantly, the inventor's preliminary data suggest that aberrant NO signaling occurs in ASD patients of different etiology and ASD mouse models, and may contribute significantly to this pathology. Nakamura et al. reported that the aberrant SNO of the dynamin-related protein 1 occurs in both the brain and blood of patients with a neurological disorder. Also, NO species can be found both in the brain and in the blood of autistic patients due to oxidative/nitrosative stress. Therefore, NO and SNO-related molecular changes occurring in the brain of ASD patients are at least partially reflected in the blood.

3) Phosphorylation (P) of amino acid residues in the proteins induced by protein kinases is also an essential PTM regulating enzyme activity in physiological and pathological conditions. This kind of PTM will be studied using phospho-proteomics. Protein phosphorylation has been shown to be involved in both neurodegenerative and neurodevelopmental disorders, including ASD. For example, a recent study identified differential expression of the mTOR and the mitogen-activated protein kinase (MAPK) pathways in 3-11 years old children affected by mild and severe idiopathic autism. They showed increased phosphorylation of a downstream target of mTOR, eIF4E, and the MAPK-interacting kinase 1. The inventor has also found amplification of the mTOR signaling in the plasma human samples as well in the Shank3 and Cntnap2 ASD mouse models, as manifested in the increased phosphorylation of the downstream target of mTOR, RPS6. Others have shown phosphorylation of the downstream targets of Protein Kinase C, β-catenin and neuroligin-4X, which are considered as autism risk molecules.

A novel approach for diagnosing diseases relies on volatile organic compounds (VOCs). Organic compounds with relatively high vapor pressure or volatility, that can be detected in blood samples, urine, skin, and/or in the exhaled breath can be an indication for diseases. Each of the many volatile compounds presents its own biochemical background. VOCs are generated in the human body by alteration of metabolic pathways, such as: liver enzymes, carbohydrate metabolism, oxidative stress, lipid metabolism, and cytochrome P450. Breath can serve as an important source for biomarkers in different cancers, neurological disorders, and other diseases. Exhaled breath contains a multitude of VOCs, such as saturated hydrocarbons, unsaturated hydrocarbons, oxygen- and sulfur-containing compounds. These compounds are produced by biological processes, including oxidative stress and inflammation in the human body, as well as by invading microorganisms. Upon their production, VOCs are excreted into the blood and then diffuse into the lungs where they are exhaled. These compounds can serve as a basis for a non-invasive, simple, inexpensive, and easy-to-use diagnostic tool.

### SUMMARY OF THE INVENTION

The present invention, in some embodiments, provides methods and kits for determining autism spectrum condition in a subject.

The present invention is based, at least in part, on the finding of protein biomarkers, including global, phospho-, and S-nitroso- biomarkers, for detecting an autism spectrum condition, such as using a blood sample. Advantageously, accuracy of at least 90% in detecting an autism spectrum condition was received using the biomarkers provided herein, and specifically, by the combination of three types of proteomics.

The present invention is further based, in part, on the finding that volatile organic compounds (VOCs) may be accurate biomarkers for detecting an autism spectrum condition in a subject, such as using breath samples. As demonstrated herein, at least twenty VOCs have been identified as biomarkers for autism spectrum condition.

According to one aspect, there is provided a method of diagnosing an autism spectrum condition in a subject, the method comprising determining in a sample obtained from the subject any one of: (i) an elevated expression level of at least one biomarker selected from Table 2; (ii) a reduced expression level of at least one biomarker selected from Table 3; (iii) phosphorylation of at least one biomarker selected from Table 4; (iv) S-nitrosylation (SNO) of at least one biomarker selected from Table 5; (v) a volatile organic compound (VOC) profile comprising at least one VOC selected from any one of Table 1a, Table 1b, Table 1c, Table 1d, Table 1e, and any combination thereof; and (vi) any combination of (i) to (v), wherein a significant change of the at least one biomarker in the sample compared to control, is indicative of the subject being afflicted with an autism spectrum condition.

According to another aspect, there is provided a method of determining a subject afflicted with an autism spectrum condition being responsive to therapy, the method comprising determining in a sample obtained from the subject any one of: (i) an elevated expression level of at least one biomarker selected from Table 2; (ii) a reduced expression level of at least one biomarker selected from Table 3; (iii) phosphorylation of at least one biomarker selected from Table 4; (iv) SNO of at least one biomarker selected from Table 5; (v) a VOC profile comprising at least one VOC selected from any one of Table 1a, Table 1b, Table 1c, Table 1d, Table 1e, and any combination thereof; and (vi) any combination of (i) to (v), wherein a significant change of the at least one biomarker in the sample compared to control, is indicative of the subject being responsive to therapy.

According to another aspect, there is provided a method of screening for a therapy suitable for treating a subject afflicted with an autism spectrum condition, the method comprising determining in a sample obtained from the subject receiving the therapy, any one of: (i) an elevated expression level of at least one biomarker selected from Table 2; (ii) a reduced expression level of at least one biomarkers selected from Table 3; (iii) phosphorylation of one or more biomarker selected from Table 4; (iv) SNO of at least one biomarker selected from Table 5; (v) a VOC profile comprising at least one VOC selected from Table 1a, Table 1b, Table 1c, Table 1d, Table 1e, and any combination thereof; and (vi) any combination of (i) to (v), wherein a significant change of the at least one biomarker in the sample compared to control, is indicative of the therapy being suitable for treating the subject afflicted with an autism spectrum condition.

According to another aspect, there is provided method for diagnosing a subject with an autism spectrum condition, the method comprising: obtaining a breath sample from the subject; and determining a VOC profile of the breath sample, wherein a significant change of the VOC profile in the breath sample compared to control, is indicative of the subject being afflicted with an autism spectrum condition.

According to another aspect, there is provided a kit comprising a reagent adapted to specifically determine at least one of: (i) expression level of at least one biomarker selected from Table 2; (ii) expression level of at least one biomarker selected from Table 3; (iii) phosphorylation of at least one biomarker selected from Table 4; (iv) SNO of at least one biomarker selected from Table 5; (v) a VOC profile comprising at least one VOC selected from any one of Table 1a, Table 1b, Table 1c, Table 1d, Table 1e, and any combination thereof; and (vi) any combination of (i) to (v).

According to another aspect, there is provided a method of diagnosing a subject with an autism spectrum condition, the method comprising, obtaining a sample selected from a breath sample and blood sample from the subject; obtaining a profile of the sample using an analytic device; inputting one or more profile into a machine learning model stored in a non-transitory memory and implemented by a processor; and diagnosing the subject as having or not having an autism spectrum condition based on the output of the machine learning model.

According to another aspect, there is provided a method of determining a biomarker signature suitable for determining autism in a subject, the method comprising, receiving a plurality of markers obtained from a plurality of subjects determined as having autism, the markers being selected from: (i) protein expression levels; (ii) phosphorylation of proteins; (iii) SNO of proteins; and (iv) VOCs profile; inputting the plurality of markers into a machine learning model stored in a non-transitory memory and implemented by a processor; and determining a biomarker signature suitable for determining autism in the subject based on the output of the machine learning model.

In some embodiments, the control is based on the at least one biomarker being determined prior to the therapy.

In some embodiments, the VOC profile comprises at least one VOC being detected in a breath sample obtained from the subject, and its corresponding quantity.

In some embodiments, the VOC profile comprises at least one VOC being selected from the group consisting of: phenol, alcohol, esters, ether, ketone, aldehyde, benzene, hydrocarbon, and any combination thereof.

In some embodiments, the VOC profile comprises at least one VOC being selected from the VOCs listed under Table 1a.

In some embodiments, the VOC profile comprises at least one VOC being selected from the VOCs listed under Table 1b.

In some embodiments, the VOC profile comprises at least one VOC being selected from the VOCs listed under Table 1c.

In some embodiments, the VOC profile comprises at least one VOC being selected from the VOCs listed under Table 1d.

In some embodiments, the VOC profile comprises at least one VOC being selected from the VOCs listed under Table 1e.

In some embodiments, the VOC profile comprises a plurality of VOCs selected from the group consisting of the VOCs listed under any one of Table 1a, Table 1b, Table 1c, Table 1d, Table 1e, and any combination thereof.

In some embodiments, the at least one biomarker is selected from Tables 2-5, and wherein the sample is selected from whole blood sample, a serum sample, or a plasma sample.

In some embodiments, the method further comprises a step of treating the subject determined as being afflicted with an autism spectrum condition with a therapeutically effective amount of therapy suitable for autism.

In some embodiments, the method comprises determining in a sample obtained from the subject: (i) an expression level of Histone H4; (ii) phosphorylation of mitochondrial Rho GTPase 1; (iii) SNO of Tuberin; and (iv) a VOC profile comprising decanal, wherein significant: increase in expression level of Histone H4, phosphorylation of mitochondrial Rho GTPase 1, SNO of Tuberin, and detection of decanal in the VOC profile, in the sample compared to control, is indicative of the subject being afflicted with an autism spectrum condition.

In some embodiments, the method comprises determining in a sample obtained from the subject: (i) an expression level of apolipoprotein C; (ii) phosphorylation of adenylate cyclase 2; (iii) SNO of apolipoprotein C-1; and (iv) a VOC profile comprising decanal, wherein significant: increase in expression level of apolipoprotein C, phosphorylation of adenylate cyclase 2, SNO of apolipoprotein C-1, and detection of decanal in the VOC profile, is indicative of the subject being afflicted with an autism spectrum condition.

In some embodiments, the kit further comprises a control or standard sample.

In some embodiments, the kit is for diagnosing autism spectrum condition in a subject.

In some embodiments, the obtaining is obtaining a protein profile of the blood sample using an analytic device, wherein the protein profile comprises one or more profiles selected from (i) expression levels; (ii) phosphorylation state; and (iii) SNO state.

In some embodiments, the obtaining is obtaining a VOC profile of the breath sample using an analytic device, wherein the VOC profile comprises one or more of the VOCs detected and its corresponding quantity.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1B include a diagram and a heatmap. (1A) Venn diagram representing the volatile organic compound (VOCs) identified in Autism spectrum disorder (ASD) and typically developing (TD) breath. (1B) Heat map analysis representing the differential relative abundance of the shared VOCs between ASD and TD. The relative abundance scale was normalized by −log 10. Each line represents one VOC.

FIG. 2 includes a graph showing a combined analysis of: (i) protein expression levels; (ii) phosphorylation of proteins; (iii) SNO of proteins; and (iv) VOCs which determined a significant clustering with accuracy, sensitivity, and specificity at 95, 97, and 92%, respectively.

FIG. 3 includes a flowchart demonstrating, as a non-limiting example, the steps for diagnosing a subject with an autism spectrum condition, according to some embodiments of the invention.

FIG. 4 includes a flowchart demonstrating, as a non-limiting example, the steps for determining a biomarker signature suitable for determining autism in a subject, according to some embodiments of the invention.

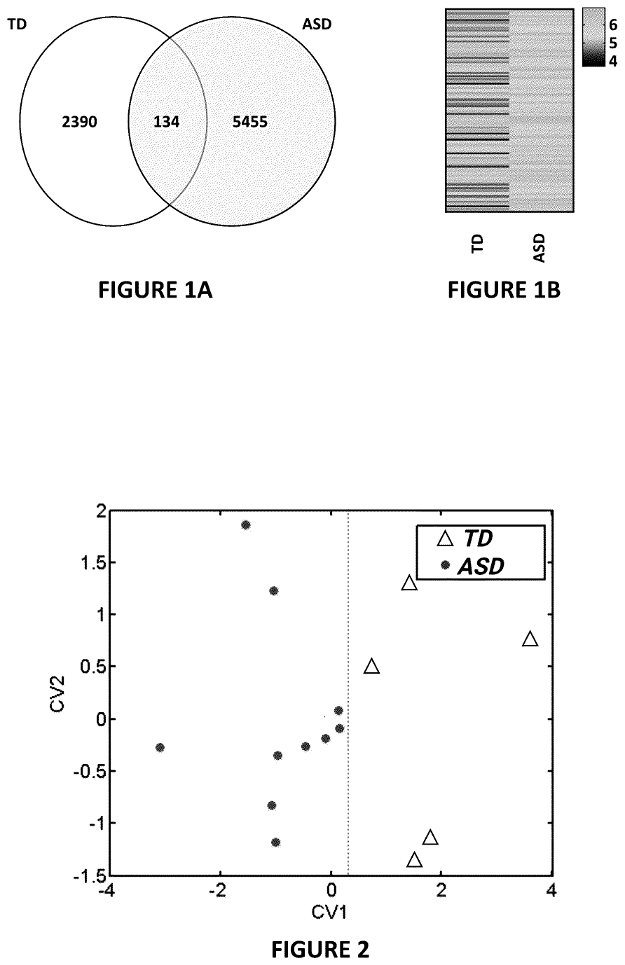

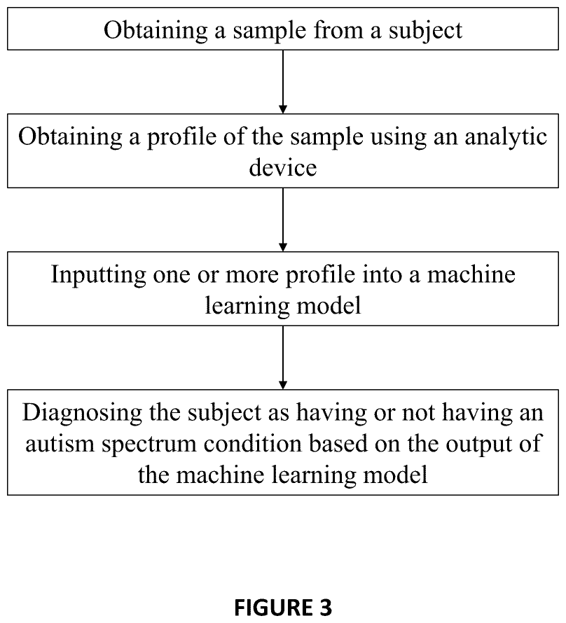

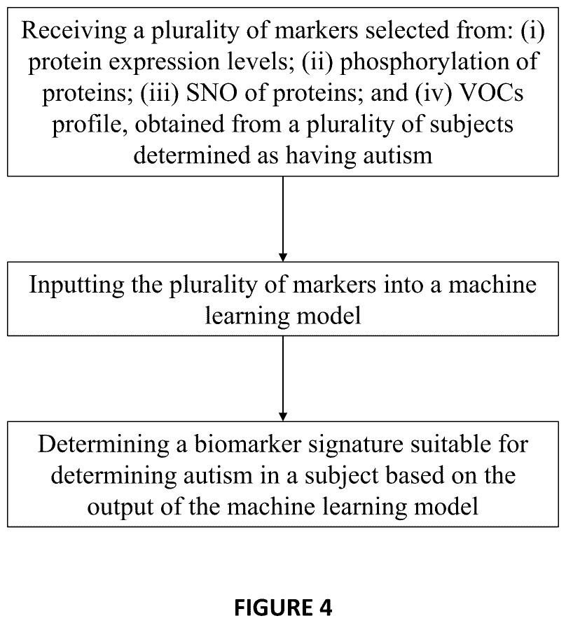

### DETAILED DESCRIPTION OF THE INVENTION

The present invention, in some embodiments, provides methods for determining an autism spectrum condition in a subject. A kit comprising reagents adapted to specifically determine one or more biomarkers is also provided.

According to some embodiments, the invention provides methods, systems and kits for screening, diagnosis or prognosis of autism spectrum disorder, including identifying subjects with a predisposition for developing an autism spectrum disorder and those most likely to respond to therapy.

According to some embodiments, the invention provides methods, systems, and kits providing a multiomics platform that relies on a combination of several sets (2, 3, or 4) comprising different sets of biomarkers, including varying expression levels of a protein signature, and PTM changes, including phosphorylation and S-nitrosylation of proteins, as well as a specific VOC signature.

As demonstrated herein (FIG. 2), a combined analysis of: (i) protein expression levels; (ii) phosphorylation of proteins; (iii) SNO of proteins; and (iv) VOCs determined a significant clustering with accuracy, sensitivity, and specificity at 95, 97, and 92%, respectively.

### Methods of Diagnosis

According to one aspect, there is provided a method of diagnosing an autism spectrum condition in a subject, the method comprising determining in a sample obtained from the subject one or more biomarker selected from: (i) an elevated expression level of one or more biomarkers selected from Table 2; (ii) a reduced expression level of one or more biomarkers selected from Table 3; (iii) phosphorylation of one or more biomarkers selected from Table 4; and (iv) S-nitrosylation (SNO) one or more biomarkers selected from Table 5; and (v) a VOC profile comprises one or more VOCs selected from Table 1a, Table 1b, Table 1c, Table 1d and Table 1e.

In some embodiments, the method comprising determining in a sample obtained from the subject at least one biomarker selected from: (i) an elevated expression level of at least one biomarker selected from Table 2; (ii) a reduced expression level of at least one biomarker selected from Table 3; (iii) phosphorylation of at least one biomarker selected from Table 4; and (iv) S-nitrosylation (SNO) of at least one biomarker selected from Table 5; and (v) a VOC profile comprising at least one VOC selected from Table 1a, Table 1b, Table 1c, Table 1d or Table 1e.

In some embodiments, a significant change of the one or more biomarker in the sample compared to control, is indicative of the subject being afflicted with an autism spectrum condition.

In some embodiments, a nonsignificant or insignificant change of the one or more biomarker in the sample compared to control, is indicative of the subject not being afflicted with an autism spectrum condition.

In some embodiments, a significant, nonsignificant, or insignificant change, is a statistically significant, nonsignificant, or insignificant change.

Statistical tools for determining significant or insignificant changes are common and would be apparent to one of ordinary skill in the art. Such tools are exemplified herein.

As used herein, the terms “nonsignificant” and “insignificant” are interchangeable. In some embodiments, at least one comprises one or more.

According to another aspect, there is provided a method for diagnosing a subject with an autism spectrum condition, the method comprising: obtaining a breath sample from the subject; and determining a VOC profile from the breath sample.

In some embodiments, a significant change of the VOC profile in the breath sample compared to control or a standard, is indicative of the subject being afflicted with an autism spectrum condition.

In some embodiments, a nonsignificant change of the VOC profile in the breath sample compared to control or a standard, is indicative of the subject not being afflicted with an autism spectrum condition.

According to another aspect, there is provided a method of screening for a therapy suitable for treating an autism spectrum condition, the method comprising determining in a sample obtained from a subject suffering from or afflicted with an autism spectrum condition, one or more biomarkers selected from: (i) an elevated expression level of one or more biomarkers selected from Table 2; (ii) a reduced expression level of one or more biomarkers selected from Table 3; (iii) phosphorylation of one or more biomarkers selected from Table 4; and (iv) S-nitrosylation (SNO) one or more biomarkers selected from Table 5; and (v) a VOC profile comprises one or more VOCs selected from Table 1a, Table 1b, Table 1c, Table 1d and Table 1e.

In some embodiments, a significant change of the one or more biomarker in the sample compared to control, is indicative of the therapy being suitable for treating an autism spectrum condition.

In some embodiments, a nonsignificant change of the one or more biomarker in the sample compared to control, is indicative of the therapy being unsuitable for treating an autism spectrum condition.

In some embodiments, the subject is a human. In some embodiments, the subject is an infant. In some embodiments, the subject is a child or a fetus. In some embodiments, the subject is a toddler. In some embodiments, the subject is a subject who is at risk of developing ASD, a subject who is suspected of having ASD, or a subject who is afflicted with ASD. Each possibility represents a separate embodiment of the invention.

In some embodiments, the VOC profile comprises one or more VOCs selected from: phenol, alcohol, esters, ether, ketone, aldehyde, benzene or hydrocarbon.

In some embodiments, the VOC profile comprises one or more VOCs selected from the VOCs listed under Table 1a.

TABLE 1a

| VOC | CAS/PubChem CID |
|---|---|
| 2,4,4-Trimethyl-1-pentanol, | 16325-63-6 |
| heptafluorobutyrate | |
| 2-Propanol, 1-methoxy- | 107-98-2 |
| [2-(2-methoxyacetyl)oxyphenyl] 3- | PubChem CID91698089 |
| methylbut-2-enoate | |
| 1,2-Propanediol dibutyrate | 50980-84-2 |
| Decanal | 112-31-2 |

In some embodiments, the VOC profile comprises one or more VOCs selected from the VOCs listing under Table 1b.

TABLE 1b

| VOC | CAS/PubChem CID |
|---|---|
| Benzeneacetic acid, | 5421-00-1 |
| (tetrahydrofuranyl)methyl ester | |
| Hydroxymethyl 2-hydroxy-2-methylpropionate | 594-61-6 |
| Hexano-dibutyrin | PubChem CID 71363740 |
| Fumaric acid, pentyl tetrahydrofurfuryl | 638-49-3 |
| ester | |
| Propanoic acid, 2-bromo-, methyl ester | 5445-17-0 |
| Ethyl ether | 60-29-7 |
| Fumaric acid, tetradecyl tetrahydrofurfuryl | PubChem CID91695664 |
| ester | |

In some embodiments, the VOC profile comprises one or more VOCs selected from the group consisting of the VOCs listing under Table 1c.

TABLE 1c

| VOC | CAS |
|---|---|
| Methyl(1-methyl-4-(1-methyl-4-nitro-2- | 13138-76-6 |
| pyrrolamido)-2-pyrrolecarboxylate) | |
| 2-Thiophenecarboxaldehyde, oxime | 29683-84-9 |

In some embodiments, the VOC profile comprises one or more VOCs selected from the group consisting of the VOCs listing under Table 1d.

TABLE 1d

| VOC | CAS |
|---|---|
| Silabenzene, 1-methyl- | 63878-65-9 |
| Benzene, 1,2,4,5-tetrafluoro-3- | 651-80-9 |
| (trifluoromethyl)- | |
| 1 ethylundecyl)-Benzene | 4534-52-5 |

In some embodiments, the VOC profile comprises one or more VOCs selected from the group consisting of the VOCs listing under Table 1e.

TABLE 1e

| VOC | CAS |
|---|---|
| 1-chloro-Decane | 1002-69-3 |
| 1-chloro-Hexadecane | 4860-03-1 |
| 2,4,6-trimethyl-Octane | 62016-37-9 |
| Nonane, 2,2,4,4,6,8,8-heptamethyl- | 909554 |

In some embodiments, the VOC profile comprises one or more of VOCs detected in a breath sample and its corresponding quantity.

In some embodiments, the VOC profile comprises a plurality of VOCs selected from the VOCs listed under any one of Table 1a, Table 1b, Table 1c, Table 1d, Table 1e, and any combination thereof.

In some embodiments, the VOC profile comprises a plurality of VOCs comprising at least one VOC selected from Table 1a, at least one VOC selected from Table 1b, at least one VOC selected from Table 1c, at least one VOC selected from Table 1d, and at least one VOC selected from Table 1e.

In some embodiments, the VOC profile comprises a plurality of VOCs. In some embodiments, the VOC profile comprises at least: 2, 3, 4, 5, 6, 7, 8, 9, 10, 11, 12, 13, 14, 15, 16, 17, 18, 19, 20 VOCs, or any value and range therebetween. Each possibility represents a separate embodiment of the invention. In some embodiments, the VOC profile comprises at most: 100, 75, 50, 45, 40, 35, 30 VOCs, or any value and range therebetween. Each possibility represents a separate embodiment of the invention.

In some embodiments, the VOC profile comprises 2-100, 10-100, 20-100, 40-100, 60-100, 80-100, 90-100, 2-10, 2-20, 2-40, 5-35, or 10-60 VOCs. Each possibility represents a separate embodiment of the invention.

According to another aspect, there is provided a method for determining a VOC profile in a breadth sample, the method comprises determining one or more VOCs selected from or listed under any one of: Table 1a, Table 1b, Table 1c, Table 1d, Table 1e, and any combination thereof, and comparing the determined VOC profile to control.

According to another aspect, there is provided a method of diagnosing an autism spectrum condition in a subject, the method comprising determining in a sample obtained from the subject one or more biomarker selected from: (i) an elevated expression level of one or more biomarkers selected from Table 2; (ii) a reduced expression level of one or more biomarkers selected from Table 3; (iii) phosphorylation of one or more biomarkers selected from Table 4; and (iv) S-nitrosylation (SNO) one or more biomarkers selected from Table 5.

In some embodiments, a significant change of the one or more biomarker in the sample compared to control, is indicative of the subject being afflicted with an autism spectrum condition.

In some embodiments, a nonsignificant change of the one or more biomarker in the sample compared to control, is indicative of the subject being not afflicted with an autism spectrum condition.

In some embodiments, a significant change of the one or more biomarker in the sample compared to control, is indicative of the subject being at increased risk of developing an autism spectrum condition.

In some embodiments, a nonsignificant change of the one or more biomarker in the sample compared to control, is indicative of the subject being at low or no risk of developing an autism spectrum condition.

In some embodiments, the sample is selected from whole blood sample, a serum sample, a plasma sample, or any combination thereof.

TABLE 2
Proteins having elevated expression levels in ASD subjects

| UniProt | |
|---|---|
| Accession no. | Protein name |
| P62805 | Histone H4 |
| P16112 | Aggrecan core protein |
| P09172 | Dopamine beta-hydroxylase; Soluble dopamine beta- |
| | hydroxylase |
| Q02487 | Desmocollin-2 |
| P40189 | Interleukin-6 receptor subunit beta |
| O75015 | Low affinity immunoglobulin gamma Fc region receptor |
| | III-B |
| P11279 | Lysosome-associated membrane glycoprotein 1 |
| Q13228 | Selenium-binding protein 1 |
| P19022 | Cadherin-2 |
| O75144 | ICOS ligand |
| P23470 | Receptor-type tyrosine-protein phosphatase gamma |
| P07339 | Cathepsin D |
| P01591 | Immunoglobulin J chain |
| Q9UEW3 | Macrophage receptor MARCO |
| Q9H4A9 | Dipeptidase 2 |
| P61626 | Lysozyme C |
| P01042 | Kininogen-1 |
| P27169 | Serum paraoxonase/arylesterase 1 |
| P02760 | Protein AMBP |
| P02790 | Hemopexin |
| P16112 | Aggrecan core protein; Aggrecan core protein 2 |
| P01008 | Antithrombin-III |
| P23470 | Receptor-type tyrosine-protein phosphatase gamma |
| P01042 | Kininogen-1; Kininogen-1 heavy chain; T-kinin; |
| | Bradykinin; Lysyl-bradykinin; Kininogen-1 light chain; |
| | Low molecular weight growth-promoting factor |

TABLE 3
Proteins having reduced expression levels in ASD subjects

| UniProt | |
|---|---|
| Accession no. | Protein name |
| P12814 | Alpha-actinin-1 |
| Q13418 | Integrin-linked protein kinase |
| P21291 | Cysteine and glycine-rich protein 1 |
| P08567 | Pleckstrin |
| P48059 | LIM and senescent cell antigen-like-containing domain |
| | protein 1 |
| P62826 | GTP-binding nuclear protein Ran |
| Q15404 | Ras suppressor protein 1 |
| P51003 | Poly(A) polymerase alpha |
| Q9H4B7 | Tubulin beta-1 chain |
| 060234 | Glia maturation factor gamma |
| Q14574 | Desmocollin-3 |
| P03973 | Antileukoproteinase |
| Q15691 | Microtubule-associated protein RP/EB family member 1 |
| Q07960 | Rho GTPase-activating protein 1 |
| Q9HBI1 | Beta-parvin |
| Q8IZP2 | Putative protein FAM10A4 |
| P55072 | Transitional endoplasmic reticulum ATPase |
| P10720 | Platelet factor 4 variant |
| P59998 | Actin-related protein 2/3 complex subunit 4 |
| Q14766 | Latent-transforming growth factor beta-binding protein 1 |
| P30086 | Phosphatidylethanolamine-binding protein 1 |
| O00151 | PDZ and LIM domain protein 1 |
| PODMV8 | Heat shock 70 kDa protein 1A |
| P31946 | 14-3-3 protein beta/alpha |
| 015145 | Actin-related protein 2/3 complex subunit 3 |
| P06744 | Glucose-6-phosphate isomerase |
| P62258 | 14-3-3 protein epsilon |
| Q9Y2X7 | ARF GTPase-activating protein GIT1 |
| P10721 | Mast/stem cell growth factor receptor Kit |
| P08758 | Annexin A5 |
| P29350 | Tyrosine-protein phosphatase non-receptor type 6 |
| P18206 | Vinculin |
| P68133 | Actin |
| P14618 | Pyruvate kinase PKM |
| P07741 | Adenine phosphoribosyltransferase |
| P28066 | Proteasome subunit alpha type-5 |
| P27797 | Calreticulin |
| P06703 | Protein S100-A6 |
| Q13790 | Apolipoprotein F |
| P04275 | von Willebrand factor |
| P04406 | Glyceraldehyde-3-phosphate dehydrogenase |
| Q13093 | Platelet-activating factor acetylhydrolase |
| P07996 | Thrombospondin-1 |
| P04075 | Fructose-bisphosphate aldolase A |
| P68871 | Hemoglobin subunit beta |
| P07195 | L-lactate dehydrogenase B chain |
| Q8IUL8 | Cartilage intermediate layer protein 2 |
| Q15063 | Periostin |
| P00918 | Carbonic anhydrase 2 |
| O14791 | Apolipoprotein L1 |
| P03973 | Antileukoproteinase |
| P04275 | von Willebrand factor; von Willebrand antigen 2 |
| P14618 | Pyruvate kinase PKM |

TABLE 4
Proteins being phosphorylated in ASD subjects

| | Uniprot | | |
|---|---|---|---|
| | Accession | | Position of |
| Sequence window | no. | Protein name | Phosphorylation |
| EKIFSEDDDYIDIVDS | P05546 | Heparin cofactor 2 | 98 |
| LSVSPTDSDVSAGNI | | | |
| (SEQ ID NO: 1) | | | |
| DDYLDLEKIFSEDDD | P05546 | Heparin cofactor 2 | 92 |
| YIDIVDSLSVSPTDS | | | |
| D (SEQ ID NO: 2) | | | |
| NAQKQWLKSEDIQR | Q08462 | Adenylate cyclase type 2 | 580 |
| ISLLFYNKVLEKEYR | | | |
| AT (SEQ ID NO: 3) | | | |
| LSGSRQDLIPSYSLG | Q9NQT8 | Kinesin-like protein | 1403 |
| SNKGRWESQQDVSQ | | KIF13B | |
| TT (SEQ ID NO: 4) | | | |
| VNRLSGSRQDLIPSY | Q9NQT8 | Kinesin-like protein | 1400 |
| SLGSNKGRWESQQD | | KIF13B | |
| VS (SEQ ID NO: 5) | | | |
| LVAENRRYQRSLPG | P19823 | Inter-alpha-trypsin | 60 |
| ESEEMMEEVDQVTL | | inhibitor heavy chain H2 | |
| YSY (SEQ ID NO: 6) | | | |
| GVTSLTAAAAFKPV | Q96HC4 | PDZ and LIM domain | 354 |
| GSTGVIKSPSWQRPN | | protein 5 | |
| QG (SEQ ID NO: 7) | | | |
| MPESLDSPTSGRPGV | Q96HC4 | PDZ and LIM domain | 341 |
| TSLTAAAAFKPVGS | | protein 5 | |
| TG (SEQ ID NO: 8) | | | |
| VTSLTAAAAFKPVG | Q96HC4 | PDZ and LIM domain | 355 |
| STGVIKSPSWQRPNQ | | protein 5 | |
| GV (SEQ ID NO: 9) | | | |
| SLDSPTSGRPGVTSL | Q96HC4 | PDZ and LIM domain | 344 |
| TAAAAFKPVGSTGV | | protein 5 | |
| IK (SEQ ID NO: 10) | | | |
| | Q8WWL7 | G2/mitotic-specific | 1192 |
| | | cyclin-B3 | |
| | P54886 | Delta-1-pyrroline-5- | 794 |
| | P54886 | carboxylate synthase; | 782 |
| | | Glutamate 5- | |
| | | kinase; Gamma-glutamyl | |
| | | phosphate reductase | |

TABLE 5
S-nitrosylation proteins found in ASD subjects

| | Uniprot |
|---|---|
| Protein name | Accession no |
| Polyubiquitin-B | POCG47 |
| Laminin subunit alpha-1 | P25391 |
| Sjoegren syndrome nuclear autoantigen 1 homolog | O43805 |
| Glyceraldehyde-3-phosphate dehydrogenase | P04406 |
| Pre-mRNA-processing factor 6 | O94906 |
| TSC2 | P49815 |

In some embodiments, the sample is a biological sample. In some embodiments, the sample is selected from: a tissue sample, a cell sample, a body fluid sample, a whole blood sample, a serum sample, a plasma sample, a saliva sample, a genital secretion sample, a sputum sample, a urine sample, a CSF sample, an amniotic fluid sample, a tear sample, a breath condensate sample, any portion or fraction thereof, or any combination thereof.

In some embodiments, the sample is a fluid sample or comprises a fluid. In some embodiments, the fluid is a biological fluid. In some embodiments, the sample is obtained or derived from the subject. In some embodiments, a blood sample comprises a peripheral blood sample and a plasma sample. In some embodiments, the sample is a plasma sample. In some embodiments, the method further comprises processing a sample obtained or derived from a subject. In some embodiments, processing comprises isolating plasma from the sample. In some embodiments, a biological fluid is selected from blood, plasma, lymph, cerebral spinal fluid, urine, feces, semen, tumor fluid, gastric fluid, exhaled air, or any combination thereof.

In some embodiments, the determining is directly in the sample. In some embodiments, the determining is in the unprocessed sample. In some embodiments, the determining is in a processed sample. In some embodiments, the method further comprises processing the sample. In some embodiments, processing comprises isolating proteins from the sample. In some embodiments, processing comprises isolating nucleic acids from the sample. In some embodiments, the processing comprises lysing cells in the sample.

In some embodiments, the method is for determining one or more VOCs in a breath sample. In some embodiments, the method further comprises the step of concentrating the exhaled breath sample.

In some embodiments, concentrating an exhaled breadth sample is by using a breath concentrator, a dehumidifying unit, or both.

The collection of a breath sample, according to the principles of the present invention, can be performed in any manner known to a person of ordinary skill in the art. In exemplary embodiments, the breath sample may be collected using a breath collector apparatus. Specifically, the breath collector apparatus is designed to collect alveolar breath samples. Exemplary breath collector apparatuses within the scope of the present invention include apparatuses approved by the American Thoracic Society/European Respiratory Society (ATS/ERS); Silkoff et al., Am. J. Respir. Crit. Care Med., 2005, 171, 912). Alveolar breath is usually collected from individuals using the off-line method.

In some embodiments, the step of determining the levels of the VOCs comprises the use of at least one technique selected from: Gas-Chromatography (GC), GC-lined Mass-Spectrometry (GC-MS), Proton Transfer Reaction Mass-Spectrometry (PTR-MS), Electronic nose device, Quartz Crystal Microbalance (QCM), or any combination thereof. Each possibility represents a separate embodiment of the invention. In one embodiment, the step of determining the levels of the VOCs comprises the use of Gas-Chromatography-Mass Spectrometry (GC-MS). Optionally, the GC-MS can be combined with solid phase microextraction (SPME).

In some embodiments, the reference levels of the VOCs include mean levels of the VOCs measured in the breath samples of subjects afflicted with a particular disease.

The determination of the level of the volatile organic compounds can be performed, according to the principles of the present invention, by the use of at least one technique including, but not limited to, Gas-Chromatography (GC), GC-lined Mass-Spectrometry (GC-MS), Proton Transfer Reaction Mass-Spectrometry (PTR-MS), Electronic nose device (E-nose), and Quartz Crystal Microbalance (QCM). Each possibility represents a separate embodiment of the invention.

Gas Chromatography (GC) linked to mass spectrometry (MS) is often used to determine the chemical identity and composition of breath VOCs (Miekisch et al. Clinica Chimica Acta, 2004, 347, 25-39). In this set-up, the GC utilizes a capillary column having characteristic dimensions (length, diameter, film thickness) as well as characteristic phase properties. The difference in the chemical properties of different molecules in a mixture allows the separation of the molecules as the sample travels through the column, wherein each molecule has a characteristic time (termed retention time) in which it passes through the column under set conditions. This allows the mass spectrometer to capture, ionize, accelerate, deflect, and detect the ionized molecules separately. The MS signal is obtained by ionization of the molecules or molecular fragments and measurement of their mass to charge ratio by comparing it to a reference collection.

Proton transfer reaction-mass spectrometry (PTR-MS) is reviewed in Lindinger et al., (Int. J. Mass Spectrom. Ion Process, 1998, 173, 191-241) and Lindinger et al., (Adv. Gas Phase Ion Chem., 2001, 4, 191-241). Briefly, PTR-MS measures VOCs that react with H30+ ions that are added from an ion source. VOCs with a proton affinity that is larger than that of water (166.5 kcal×mol″1) undergo a proton-transfer reaction with the H30+ ions as follows: H30++R→RH++H20. At the end of the drift tube reactor, a fraction of the ions is sampled by a quadrupole mass spectrometer, which measures the H30+ and RH+ ions. The ion signal at a certain mass is linearly dependent on the concentration of the precursor VOC in the sample air. In PTR-MS only the mass of VOCs is determined, causing some ambiguity in the identity of the VOCs. Thus, this technique does not allow a separate detection of different VOCs having the same mass. Further overlap of ion masses is caused by a limited degree of ion fragmentation and ion clustering in the drift tube.

Quartz Crystal Microbalance (QCM) is a piezoelectric-based device which can measure very small mass changes, mostly down to few nanograms. Briefly, QCM works by sending an electrical signal through a gold-plated quartz crystal, which causes vibrations in the crystal at a specific resonant frequency measured by the QCM.

Electronic nose devices perform odor detection through the use of an array of broadly cross-reactive sensors in conjunction with pattern recognition methods (see Rock et al, Chem. Rev., 2008, 108, 705-725). In contrast to the “lock-and-key” approach, each sensor in the electronic nose device is broadly responsive to a variety of odorants. In this architecture, each analyte produces a distinct fingerprint from the array of broadly cross-reactive sensors. This allows to considerably widen the variety of compounds to which a given matrix is sensitive, to increase the degree of component identification and, in specific cases, to perform an analysis of individual components in complex multi-component (bio) chemical media. Pattern recognition algorithms can then be used to obtain information on the identity, properties and concentration of the vapor exposed to the electronic nose device.

The terms “expression” and “expression levels” are used herein interchangeably and refer to the amount of a gene product present in the sample. In some embodiments, determining comprises normalization of expression levels. Determining of the expression level of the biomarker can be performed by any method known in the art. Methods of determining protein expression include, for example, western blot, antibody arrays, immunoblotting, immunohistochemistry, flow cytometry (FACS), enzyme-linked immunosorbent assay (ELISA), proximity extension assay (PEA), proteomics arrays, proteome sequencing, flow cytometry (CyTOF), multiplex assays, mass spectrometry and chromatography. In some embodiments, determining protein expression levels comprises ELISA. In some embodiments, determining protein expression levels comprises protein array hybridization. In some embodiments, determining protein expression levels comprises mass-spectrometry quantification. Methods of determining mRNA expression include, for example, RT-PCR, quantitative PCR, real-time PCR, microarrays, northern blotting, in situ hybridization, next generation sequencing, and massively parallel sequencing.

In some embodiments, a gene product includes a transcript (e.g., a messenger RNA (mRNA)), a proteinaceous product, or both.

In some embodiments, the method of the present invention comprises an analyzing step comprising determining an expression pattern of the at least one biomarker, as disclosed herein. In some embodiments, the determining comprises calculating the change in expression of the at least one marker (e.g., of Tables 1a-1e, and Tables 2-3).

In some embodiments, the pattern is analyzed with a pattern recognition analyzer which utilizes various algorithms including, but not limited to, artificial neural networks, multi-layer perception (MLP), generalized regression neural network (GRNN), fuzzy inference systems (FIS), self-organizing map (SOM), radial bias function (RBF), genetic algorithms (GAS), neuro-fuzzy systems (NFS), adaptive resonance theory (ART) and statistical methods including, but not limited to, principal component analysis (PCA), partial least squares (PLS), multiple linear regression (MLR), principal component regression (PCR), discriminant function analysis (DFA) including linear discriminant analysis (LDA), and cluster analysis including nearest neighbor. Each possibility represents a separate embodiment of the invention.

In some embodiments, a phosphorylated residue on a protein may be reacted with a detection entity, which may be, for example, fluorescent, radioactive, electron-dense, able to bind to a signaling entity or a binding partner in order to produce a signal, etc.

In some embodiments, a nitrosylated or otherwise oxidized moiety on a protein may be reacted with a detection entity, which may be, for example, fluorescent, radioactive, electron-dense, able to bind to a signaling entity or a binding partner in order to produce a signal, etc.

In some embodiments, the method of the present invention comprises determining at least one control marker, e.g., expression of at least one control marker. In some embodiments, the method further comprises determining expression level(s) of a control marker in the sample. In some embodiments, the expression of the at least one marker is normalized to expression of the control. In some embodiments, the control is used to confirm the quality of the sample, the data produced from the sample, or both. In some embodiments, the control is a housekeeping gene/protein. Housekeeping genes/proteins are well known in the art and any such gene/protein may be used as a control. Generally, housekeeping genes/proteins would be apparent to one of ordinary skill in the art as constitutively expressed, easily measured, having known and/or predictable expression trend/pattern, and play a role in an essential cellular function.

According to some embodiments, a control sample may be obtained from a reference group comprising subjects which are not afflicted with ASD (negative control). The control sample, according to the principles of the present invention in some embodiments, is obtained from at least one subject, preferably a plurality of subjects. A set of control samples from subjects who are not afflicted with ASD may be stored as a reference collection of data.

In some embodiments, the method further comprises treating a subject determined as being afflicted with an autism spectrum condition with a therapy suitable for autism.

In some embodiments, therapy suitable for autism is selected from: behavioral therapy, developmental therapy, educational therapy, social-relational therapy, physiological therapy, complementary and alternative therapy, or any combination thereof.

In some embodiments, behavioral therapy comprises applied behavior analysis (ABA). In some embodiments, ABA comprises discrete trial training (DTT), pivotal response training (PRT), or both.

In some embodiments, a developmental therapy comprises speech and language therapy, occupational therapy, or both. In some embodiments, occupational therapy comprises sensory integration therapy, physical therapy, or both.

In some embodiments, educational therapy comprises treatment and education of autistic and related communication-handicapped children (TEACCH).

In some embodiments, social-relational therapy comprises developmental, individual differences, relationship-based therapy (e.g., “floor time”), relationship development intervention (RDI), social stories, social skill groups, or any combination thereof.

In some embodiments, psychological therapy comprises cognitive-behavior therapy (CBT).

Methods for autism therapy, as described hereinabove, are common and would be apparent to one of ordinary skill in the art, see for example Hyman et al., Pediatrics, (2020)).

As used herein, the terms “administering”, “administration”, and the like refer to any method which, in sound medical practice, delivers a composition containing an active agent to a subject in such a manner as to provide a therapeutic effect.

The dosage administered will be dependent upon the age, health, and weight of the recipient, kind of concurrent treatment, if any, frequency of treatment, and the nature of the effect desired.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life.

### Kits

According to another aspect, there is provided a kit comprising a reagent adapted to specifically determine at least one biomarker selected from: (i) a VOC profile comprising at least one VOC being selected from: Table 1a, Table 1b, Table 1c, Table 1d or Table 1e; (ii) expression level of at least one biomarker selected from Table 2; (iii) expression level of at least one biomarker selected from Table 3; (iv) phosphorylation of at least one biomarker selected from Table 4; (v) S-nitrosylation (SNO) of at least one biomarker selected from Table 5, and (vi) any combination of (i) to (vi).

In some embodiments, the kit is for diagnosing autism spectrum condition in a subject.

Reagents for detecting protein expression are well known in the art and include antibodies, protein binding arrays, protein binding proteins, protein binding aptamers and protein binding RNAs. Any reagent capable of binding specifically to the factor can be employed. As used herein, the terms “specific” and “specifically” refer to the ability to quantify the expression of one target to the exclusion of all other targets. Thus, for non-limiting example, an antibody that is specific to a target will bind to that target and no other targets. In some embodiments, the reagent is an antibody. In some embodiments, binding to a target and no other targets is binding measurable to a target and to no other targets. In some embodiments, binding to a target and no other targets is binding significantly to a target and no other targets. Reagents for detecting specific mRNAs are also well known in the art and include, for example, microarrays, primers, hybridization probes, and RNA-binding proteins. Any such reagent may be used. In some embodiments, the reagent is a primer. In some embodiments, the reagent is a pair of primers specific to the biomarker.

In some embodiments, the kit further comprises at least one reagent adapted to specifically determine the expression level of a control. In some embodiments, the control is a control such as described herein. It will be understood that if the kit comprises reagents for determining protein expression of the biomarker, then the reagent for determining expression of the control would also determine protein expression. In some embodiments, the reagent for determining expression of the biomarker (e.g., in a sample obtained or derived from a subject) and the reagent for determining expression of the control are the same type of reagent. In some embodiments, the kit further comprises detectable tag or label. In some embodiments, the reagents are hybridized or attached to the label. In some embodiments, the kit further comprises a secondary reagent for detection of the specific reagents. In some embodiments, the secondary reagents are non-specific and will detect all or a subset of the specific reagents. In some embodiments, the secondary reagents are secondary antibodies. In some embodiments, the secondary reagents are detectable. In some embodiments, the secondary reagents comprise a tag or label. In some embodiments, the tag or label is detectable. In some embodiments, a detectable molecule comprises a detectable moiety. Examples of detectable moieties include fluorescent moieties, dyes, bulky groups and radioactive moieties.

In some embodiments, the reagent comprises an agent having specific or increased binding affinity to a biomarker as disclosed herein. In some embodiments, the agent is a binding protein. In some embodiments, the agent is an antibody. In some embodiments, the agent is an antagonist. In some embodiments, the agent has specific or increased binding affinity to a phosphorylated isoform or polymorph of the biomarker disclosed herein. In some embodiments, the agent comprises a nucleic acid. In some embodiments, the agent is an oligonucleotide. In some embodiments, the agent is a nucleic acid-based probe. In some embodiments, the kit comprises oligonucleotides suitable for exponential amplification of a transcript of a biomarker as disclosed herein, e.g., as listed under Tables 2 and/or 3. In some embodiments, the kit comprises oligonucleotides, primers, etc. suitable for PCR amplification of a transcript or a complementary DNA (cDNA) thereof of a biomarker as disclosed herein, e.g., as listed under Tables 2 and/or 3. In some embodiments, the kit comprises reagents suitable for reverse transcription.

In some embodiments, the agent does bind, has high binding affinity to a phosphorylated biomarker being listed under Table 4. In some embodiments, the agent does not bind, has low binding affinity, or no binding affinity to a non-phosphorylated biomarker being listed under Table 4.

In some embodiments, the kit further comprises a control sample or a standard sample. The terms “control” and “standard” are used herein interchangeably, and comprises or refers to any control sample as disclosed herein.

In some embodiments, the kits further comprise a breath concentrator, a dehumidifying unit, or both.

Breath concentrators that are within the scope of the present invention include, but are not limited to, (i) Solid Phase Microextraction (SPME)—The SPME technique is based on a fiber coated with a liquid (polymer), a solid (sorbent), or combination thereof. The fiber coating extracts the compounds from the sample either by absorption (where the coating is liquid) or by adsorption (where the coating is solid). Non-limiting examples of coating polymers include polydimethylsiloxane, polydimethylsiloxane-divinylbenzene and polydimethylsiloxane-carboxen. (ii) Sorbent Tubes-Sorbent tubes are typically made of glass and contain various types of solid adsorbent material (sorbents). Commonly used sorbents include activated charcoal, silica gel, and organic porous polymers such as Tenax and Amberlite XAD resins. Sorbent tubes are attached to air sampling pumps for sample collection. A pump with a calibrated flow rate in ml/min draws a predetermined volume of air through the sorbent tube. Compounds are trapped onto the sorbent material throughout the sampling period. This technique was developed by the US National Institute for Occupational Safety and Health (NIOSH); (iii) Cryogenic Concentrations—Cryogenic condensation is a process that allows recovery of volatile organic compounds (VOCs) for reuse. The condensation process requires very low temperatures so that VOCs can be condensed. Traditionally, chlorofluorocarbon (CFC) refrigerants have been used to condense the VOCs. Currently, liquid nitrogen is used in the cryogenic (less than −160° C.) condensation process.

In some embodiments, the kit further comprises a solution for rendering a protein susceptible to binding. In some embodiments, the kit further comprises a solution for lysing cells. In some embodiments, the kit further comprises a solution for isolating plasma from blood. In some embodiments, the kit further comprises a solution for purification of proteins.

In some embodiments, a reagent is attached to linked to a solid support. In some embodiments, the reagent is non-natural. In some embodiments, the reagent is artificial. In some embodiments, the reagent is in a non-organic solution. In some embodiments, the reagent is ex vivo. In some embodiments, the reagent is in a vial. In some embodiments, the solid support is non-organic. In some embodiments, the solid support is artificial. In some embodiments, the solid support is an array. In some embodiments, the solid support is a chip. In some embodiments, the solid support is a bead.

Autism spectrum disorders are generally characterized as one of five disorders coming under the umbrella of Pervasive Developmental Disorders (PDD). The five disorders under PDD include autism (classical autism), Asperger's Syndrome, Rett's Syndrome, childhood disintegrative disorder, and pervasive developmental disorder not otherwise specified (PDD-NOS).

In certain embodiments, the autism is non-syndromic autism. In some embodiments, the presence or increased risk of developing other types of autism spectrum disorders may be characterized.

The methods and kits of the invention may further be used for diagnosing or predicting increased risk of developing a genetic syndrome or idiopathic reason linked to autism, thereby determining whether the subject is afflicted with, or at increased risk of developing, syndromic autism or non-syndromic autism or another autism spectrum disorder.

Genetic disorders that are generally linked to autism include, for example, genetic mutations including SHANK3, CNTNAP2, NLGN3, Angelman syndrome, Prader-Willi syndrome, 15ql 1-ql3 duplication, fragile X syndrome, fragile X premutation, deletion of chromosome 2q, XYY syndrome, Smith-Lemli-Opitz syndrome, Apert syndrome, mutations in the ARX gene, De Lange syndrome, Smith-Magenis syndrome, Williams syndrome, Noonan syndrome, Down syndrome, velo-cardio-facial syndrome, myotonic dystrophy, Steinert disease, tuberous sclerosis, Duchenne's disease, Timothy syndrome, lOp terminal deletion, Cowden syndrome, 45,X/46,XY mosaicism, Myhre syndrome, Sotos syndrome, Cohen syndrome, Goldenhar syndrome, Joubert syndrome, Lujan-Fryns syndrome, Moebius syndrome, hypomelanosis of Ito, neurofibromatosis type 1, CHARGE syndrome, and HEADD syndrome.

As used herein, the term “diagnosis” means detecting a disease or disorder or determining the stage, severity or degree of a disease or disorder, distinguishing a disease from other diseases including those diseases that may feature one or more similar or identical symptoms, monitoring disease progression or relapse, as well as assessment of treatment efficacy and/or relapse of a disease, disorder or condition, as well as selecting a therapy and/or a treatment for a disease, optimization of a given therapy for a disease, monitoring the treatment of a disease, and/or predicting the suitability of a therapy for specific patients or subpopulations or determining the appropriate dosing of a therapeutic product in patients or subpopulations. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. The diagnostic methods may be used independently, or in combination with other diagnosing and/or staging methods known in the medical art for a particular disease or disorder, e.g., HCC.

The term “prognosis” as used herein refers to a prediction of the probable course and outcome of a clinical condition or disease. A prognosis is usually made by evaluating factors or symptoms of a disease that are indicative of a favorable or unfavorable course or outcome of the disease. The phrases “prognosticating” and “determining the prognosis” are used interchangeably and refer to the process by which the skilled artisan can predict the course or outcome of a condition in a patient. The skilled artisan will understand that the term “prognosis” refers to an increased probability that a certain course or outcome will occur; that is, that a course or outcome is more likely to occur in a patient exhibiting a given condition, when compared to those individuals not exhibiting the condition. The terms “favorable prognosis” and “positive prognosis,” or “unfavorable prognosis” and “negative prognosis” as used herein are relative terms for the prediction of the probable course and/or likely outcome of a condition or a disease. A favorable or positive prognosis predicts a better outcome for a condition than an unfavorable or negative prognosis. In a general sense, a “favorable prognosis” is an outcome that is relatively better than many other possible prognoses that could be associated with a particular condition, whereas an unfavorable prognosis predicts an outcome that is relatively worse than many other possible prognoses that could be associated with a particular condition. Typical examples of a favorable or positive prognosis include a better than average cure rate, a lower propensity for metastasis, a longer than expected life expectancy, differentiation of a benign process from a cancerous process, and the like. For example, a positive prognosis is one where a patient has a 50% probability of being cured of a particular cancer after treatment, while the average patient with the same cancer has only a 25% probability of being cured.

### Machine Learning Methods and Systems

Although embodiments of the invention are not limited in this regard, discussions utilizing terms such as, for example, “processing”, “computing”, “calculating”, “determining”, “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

An apparatus, system and method according to embodiments of the invention may determine a biomarker signature suitable for determining autism in a subject and based on the identified changes of proteins and VOCs, determine a biomarker signature suitable for determining autism in a subject. In some embodiments, the markers being selected from (i) protein expression levels; (ii) phosphorylation of proteins; (iii) SNO of proteins; and (iv) VOCs.

Embodiments of the invention may include an article such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein. For example, an article may include a storage medium, computer-executable instructions and a controller.

Some embodiments may be provided in a computer program product that may include a non-transitory machine-readable medium, stored thereon instructions, which may be used to program a computer, controller, or other programmable devices, to perform methods as disclosed herein. Embodiments of the invention may include an article such as a computer or processor non-transitory readable medium, or a computer or processor non-transitory storage medium, such as for example a memory, a disk drive, or a USB flash memory, encoding, including or storing instructions, e.g., computer-executable instructions, which when executed by a processor or controller, carry out methods disclosed herein. The storage medium may include, but is not limited to, any type of disk including, semiconductor devices such as read-only memories (ROMs) and/or random access memories (RAMs), flash memories, electrically erasable programmable read-only memories (EEPROMs) or any type of media suitable for storing electronic instructions, including programmable storage devices.

A system according to embodiments of the invention may include components such as, but not limited to, a plurality of central processing units (CPU) or any other suitable multi-purpose or specific processors or controllers (e.g., controllers similar to controller 105), a plurality of input units, a plurality of output units, a plurality of memory units, and a plurality of storage units. A system may additionally include other suitable hardware components and/or software components. In some embodiments, a system may include or may be, for example, a personal computer, a desktop computer, a laptop computer, a workstation, a server computer, a network device, or any other suitable computing device.

### General

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include immunological, chemical, molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Strategies for Protein Purification and Characterization—A Laboratory Course Manual” CSHL Press (1996); all of which are incorporated by reference. Other general references are provided throughout this document.

### Methods

MS Analysis of Samples Collected from ASD and TD Children (A Four-Way Multi-Omics Platform)

Global Proteomics. The processing of plasma samples for global proteomics are carried out. Briefly, 14 abundant serum/plasma proteins are depleted and the samples undergo tryptic digestion and desalting. The resulting peptides are analyzed using nanoflow liquid chromatography (nanoAcquity) coupled with high resolution/high mass accuracy mass spectrometry (Q Exactive HFX). Raw data are processed using MaxQuant software. The data is searched with the Andromeda search engine against the human SwissProt proteome database appended with common lab protein contaminants. The Label-Free Quantification (LFQ) intensities are calculated and used for further calculations using Perseus software. Decoy hits are filtered out and common contaminates are labeled. The LFQ intensities are log-transformed and only proteins that have at least 2 or 3 valid values are taken into account.

Phospho-Proteomics. The protein-depleted, tryptic-digested and desalted plasma samples prepared for global proteomics are used for the analysis of phospho-proteomics. The phospho-proteomics analysis of the plasma samples are performed as described previously. Briefly, the samples are subjected to an IMAC phospho-enrichment on a Bravo automated sample preparation robot. The resulting peptides are analyzed using nanoAcquity coupled to Q Exactive HFX. Each sample is analyzed on the instrument separately in a random order in discovery mode. Raw data are processed using MaxQuant software. The data are searched with the Andromeda search engine against the human SwissProt proteome database appended with common lab protein contaminants and the following modifications: Carbamidomethylation of Cys as a fixed modification and oxidation of Met, protein N-terminal acetylation, and phosphorylation of Ser-Thr-Tyr as variable modifications. The phospho-site intensities are determined and used for further calculations using Perseus software. Decoy hits are filtered out and information about the linear motifs is added (from PhosphoSitePlus). The common contaminants are labeled with a ‘+’ sign in the relevant column. The site intensities are log-transformed and only sites with at least two valid values in at least one experimental group are kept. The data are then normalized by subtracting the median, and the remaining missing values are imputed by a low constant (−6).

SNO-proteomics. This procedure called SNOTRAP is carried out according to the technique that present inventor has developed and recently used in a mouse brain. Briefly, SNOTRAP labeling stock solutions are added to the samples used for the analysis of global proteome. The SNO proteins are separated using Streptavidin agarose beads and trypsinized. The digested peptides are analyzed using nanoAcquity coupled to Q Exactive HFX. The MS/MS spectra are searched against the Human SwissProt proteome database.

Different modifications of oxidation of Methionine, deamidation of Asparagine, and fixed modification of Cysteine carbamidomethylation are included in the data processing. Raw data are processed with MaxQuant software.

Breath samples are collected from individuals with ASD and TD subjects. The patients were in fast before breath samples collection. The samples were acquired employing the BioVOC™ breath sampler device (Markes International, UK). During breath sampling, the patient exhaled normally through a disposable mouthpiece until totally emptying the lungs.

The Thermal Desorption (TD) Tube was introduced into a Multi-tube thermal desorbed made by Markes (UK), model TD-100-xr. The TD tube was heated for 10 minutes to a temperature of 250° ° C., at a trap flow of 50 ml/min to a cold trap at a temperature of 10° C. Then, the cold trap is heated to a temperature of 300° C. for 3 minutes at a flow of 50 ml/min, with a split flow of 5 ml/min, giving a split ratio of 1:11 when the GC column flow is 0.5 ml/min. The analysis is performed using an Agilent GCMS instrument with GC Model 7890 and MSD Model 5977B. The TD sample was inserted through a GC injector (without liner) at a Helium constant flow of 0.5 ml/min and injector temperature of 200° C., into a BPX5 capillary GC column made by SGE cat number of 054140 with a length of 20 m in diameter (ID) of 0.18 mm and film thickness of 0.18 μm. The separation was performed after performing a temperature gradient starting at 40° ° C. for 5 minutes and increasing at a rate of 5° C./min to 100° C. (0 min) and from there increasing at a rate of 10° C./min to 250° C. (1.5 min). The sample separated in GC is inserted into a mass detector via a transfer line at a temperature of 260° ° C. without solvent delay. The molecules are detected in Scan Mode in the m/z range of 35-600. The data analysis was performed using Agilent Mass Hunter software. In the first stage, deconvolution was performed using the Mass Hunter Unknown software. From there the results were transferred to EXL, where they were processed in a pivotable.

### Data and Quantitative Analysis

For systems biology analysis of Biological Processes (BP), and pathway maps, the inventor uploaded the lists of all volatiles into MetaCore from Thomson Reuter (MetaCore™ version 6.34 build 69200). The Benjamini-Hochberg correction was used on the p-value to generate FDR, and terms with FDR values below 0.05 were accepted.

By conducting a T-test with Benforroni correction for all VOCs, a specific list of VOCs (metabolites) were found as significant. Furthermore, these metabolites may be classified based on their chemical families.

### Example 1

### First Cohort Analysis According to the Method of the Invention

A first cohort of 10 subjects afflicted with autism and 5 healthy volunteers (6-14 years old) revealed the biomarker signatures as listed under Tables 6-10 below.

TABLE 6
The most significant VOCs being classified based on their chemical family

| Phenols and | | Ketones and | | hydrocarbons |
|---|---|---|---|---|
| alcohols | Esters and ethers | aldehydes | Benzenes | and others |
| 2,4,4- | Benzeneacetic acid, | Methyl(1-methyl-4- | Silabenzene, | 1-chloro- |
| Trimethyl-1- | (tetrahydrofurany1) | (1-methyl-4-nitro-2- | 1-methyl- | Decane, |
| pentanol, | methyl ester | pyrrolamido)-2- | | |
| heptafluorobutyrate | | pyrrolecarboxylate) | | |
| 2-Propanol, 1- | Hydroxymethyl 2- | 2- | Benzene, | , 1-chloro- |
| methoxy- | hydroxy-2- | Thiophenecarboxaldehyde, | 1,2,4,5- | Hexadecane |
| | methylpropionate | oxime | tetrafluoro-3- | |
| | | | (trifluoromethyl)- | |
| 1,2- | Hexano-dibutyrin | | 1 ethylundecyl)- | 2,4,6- |
| Benzenediol, | | | Benzene | trimethyl- |
| O,O′-(3- | | | | Octane, |
| methylbut-2- | | | | |
| enoyl)-O′- | | | | |
| methoxyacetyl- | | | | |
| 1,2- | Fumaric acid, | | | Nonane, |
| Propanediol | pentyl | | | 2,2,4,4,6,8, |
| dibutyrate | tetrahydrofurfuryl | | | 8- |
| | ester | | | heptamethyl- |
| | Propanoic acid, 2- | | | |
| | bromo-, methyl | | | |
| | ester | | | |
| | Ethyl ether | | | |
| | Fumaric acid, | | | |
| | tetradecyl | | | |
| | tetrahydrofurfuryl | | | |
| | ester | | | |

TABLE 7
Proteins having elevated expression levels in ASD subjects

| ID | P-value | FC (ASD/C) | Protein name |
|---|---|---|---|
| P62805 | 0.02017283 | 9.830937141 | Histone H4 |
| P16112 | 0.001279 | 5.097575942 | Aggrecan core protein |
| P09172 | 0.0487652 | 4.982500419 | Dopamine beta-hydroxylase; Soluble dopamine |
| | | | beta-hydroxylase |
| Q02487 | 0.00901975 | 4.646527525 | Desmocollin-2 |
| P40189 | 0.03437318 | 4.334055135 | Interleukin-6 receptor subunit beta |
| O75015 | 0.04639708 | 2.787992508 | Low affinity immunoglobulin gamma Fc region |
| | | | receptor III-B |
| P11279 | 0.0350164 | 2.730205801 | Lysosome-associated membrane glycoprotein 1 |
| Q13228 | 0.02861605 | 2.728794117 | Selenium-binding protein 1 |
| P19022 | 0.02077034 | 2.631831706 | Cadherin-2 |
| O75144 | 0.02154067 | 2.622304445 | ICOS ligand |
| P23470 | 0.02344961 | 2.568553961 | Receptor-type tyrosine-protein phosphatase gamma |
| P07339 | 0.01515872 | 2.124696274 | Cathepsin D |
| P01591 | 0.02075588 | 1.912525234 | Immunoglobulin J chain |
| Q9UEW3 | 0.03372895 | 1.902304636 | Macrophage receptor MARCO |
| Q9H4A9 | 0.03942606 | 1.852713076 | Dipeptidase 2 |
| P61626 | 0.02816384 | 1.703413184 | Lysozyme C |
| P01042 | 0.00602051 | 1.658065456 | Kininogen-1 |
| P27169 | 0.01021161 | 1.571991713 | Serum paraoxonase/arylesterase 1 |
| P02760 | 0.02706635 | 1.546192249 | Protein AMBP |
| P02790 | 0.00510289 | 1.51449669 | Hemopexin |
| P16112 | 0.00219965 | 5.097576 | Aggrecan core protein; Aggrecan core protein 2 |
| P23470 | 0.00484876 | 2.568554 | Receptor-type tyrosine-protein phosphatase gamma |
| P01042 | 0.00705073 | 1.658065 | Kininogen-1; Kininogen-1 heavy chain; T- |
| | | | kinin; Bradykinin; Lysyl-bradykinin; Kininogen-1 |
| | | | light chain; Low molecular weight growth- |
| | | | promoting factor |

TABLE 8
Proteins having reduced expression levels in ASD subjects

| | | FC | |
|---|---|---|---|
| ID | P-val | (ASD/C) | Protein names |
| P12814 | 0.021995182 | 0.069381 | Alpha-actinin-1 |
| Q13418 | 0.007195168 | 0.087685 | Integrin-linked protein kinase |
| P21291 | 0.005182859 | 0.096837 | Cysteine and glycine-rich protein 1 |
| P08567 | 0.027126652 | 0.117409 | Pleckstrin |
| P48059 | 0.016444496 | 0.125998 | LIM and senescent cell antigen-like-containing |
| | | | domain protein 1 |
| P62826 | 0.016685412 | 0.143287 | GTP-binding nuclear protein Ran |
| Q15404 | 0.015339901 | 0.16666 | Ras suppressor protein 1 |
| P51003 | 0.027924152 | 0.167296 | Poly(A) polymerase alpha |
| Q9H4B7 | 0.026634488 | 0.17345 | Tubulin beta-1 chain |
| O60234 | 0.012865853 | 0.176746 | Glia maturation factor gamma |
| Q14574 | 0.012727676 | 0.176968 | Desmocollin-3 |
| P03973 | 0.001509284 | 0.199246 | Antileukoproteinase |
| Q15691 | 0.025752021 | 0.192241 | Microtubule-associated protein RP/EB family |
| | | | member 1 |
| Q07960 | 0.012703771 | 0.191773 | Rho GTPase-activating protein 1 |
| Q9HBI1 | 0.025604385 | 0.19111 | Beta-parvin |
| Q8IZP2 | 0.02383003 | 0.200206 | Putative protein FAM10A4 |
| P55072 | 0.012931059 | 0.206684 | Transitional endoplasmic reticulum ATPase |
| P10720 | 0.022127001 | 0.209499 | Platelet factor 4 variant |
| P59998 | 0.039627587 | 0.209104 | Actin-related protein 2/3 complex subunit 4 |
| Q14766 | 0.036190264 | 0.213278 | Latent-transforming growth factor beta-binding |
| | | | protein 1 |
| P30086 | 0.041120643 | 0.214568 | Phosphatidylethanolamine-binding protein 1 |
| O00151 | 0.025960254 | 0.216207 | PDZ and LIM domain protein 1 |
| P0DMV8 | 0.044421349 | 0.221706 | Heat shock 70 kDa protein 1A |
| P31946 | 0.016985731 | 0.223483 | 14-3-3 protein beta/alpha |
| O15145 | 0.015949094 | 0.225868 | Actin-related protein 2/3 complex subunit 3 |
| P06744 | 0.038350025 | 0.226678 | Glucose-6-phosphate isomerase |
| P62258 | 0.039081305 | 0.241277 | 14-3-3 protein epsilon |
| Q9Y2X7 | 0.043572382 | 0.243399 | ARF GTPase-activating protein GIT1 |
| P10721 | 0.01217467 | 0.243687 | Mast/stem cell growth factor receptor Kit |
| P08758 | 0.041297334 | 0.277315 | Annexin A5 |
| P29350 | 0.014884212 | 0.277508 | Tyrosine-protein phosphatase non-receptor type 6 |
| P18206 | 0.00903881 | 0.35009 | Vinculin |
| P68133 | 0.049744797 | 0.360259 | Actin |
| P14618 | 0.001687432 | 0.365721 | Pyruvate kinase PKM |
| P07741 | 0.04936679 | 0.376746 | Adenine phosphoribosyltransferase |
| P28066 | 0.023701987 | 0.397129 | Proteasome subunit alpha type-5 |
| P27797 | 0.036960806 | 0.415384 | Calreticulin |
| P06703 | 0.037630785 | 0.423431 | Protein S100-A6 |
| Q13790 | 0.006855443 | 0.472859 | Apolipoprotein F |
| P04275 | 0.004597415 | 0.486331 | von Willebrand factor |
| P04406 | 0.014793016 | 0.537179 | Glyceraldehyde-3-phosphate dehydrogenase |
| Q13093 | 0.009961592 | 0.546634 | Platelet-activating factor acetylhydrolase |
| P07996 | 0.042290539 | 0.556575 | Thrombospondin-1 |
| P04075 | 0.023119929 | 0.558715 | Fructose-bisphosphate aldolase A |
| P68871 | 0.022587285 | 0.584625 | Hemoglobin subunit beta |
| P07195 | 0.015628219 | 0.631921 | L-lactate dehydrogenase B chain |
| Q8IUL8 | 0.021813246 | 0.650127 | Cartilage intermediate layer protein 2 |
| Q15063 | 0.009413515 | 0.65642 | Periostin |
| P00918 | 0.039589361 | 0.65883 | Carbonic anhydrase 2 |
| 014791 | 0.002199647 | 0.613013 | Apolipoprotein L1 |
| P03973 | 0.004848763 | 0.219474 | Antileukoproteinase |
| P04275 | 0.004848763 | 0.373116 | von Willebrand factor; von Willebrand antigen 2 |
| P14618 | 0.007050729 | 0.325667 | Pyruvate kinase PKM |
| P01008 | 0.00484876 | 0.19759 | Antithrombin-III |

TABLE 9
Proteins being phosphorylated in ASD subjects

| Sequence window | Protein | Protein names | Position | p-val | FC |
|---|---|---|---|---|---|
| EKIFSEDDDYIDIV | P05546 | Heparin cofactor 2 | 98 | 0.009876 | 8.910823 |
| DSLSVSPTDSDVS | | | | | |
| AGNI (SEQ ID NO: | | | | | |
| 1) | | | | | |
| DDYLDLEKIFSED | P05546 | Heparin cofactor 2 | 92 | 0.001625 | 8.017458 |
| DDYIDIVDSLSVSP | | | | | |
| TDSD (SEQ ID NO: | | | | | |
| 2) | | | | | |
| NAQKQWLKSEDI | Q08462 | Adenylate cyclase type | 580 | 0.0468 | 7.631837 |
| QRISLLFYNKVLE | | 2 | | | |
| KEYRAT (SEQ ID | | | | | |
| NO: 3) | | | | | |
| LSGSRQDLIPSYSL | Q9NQT8 | Kinesin-like protein | 1403 | 0.017431 | 4.716845 |
| GSNKGRWESQQD | | KIF13B | | | |
| VSQTT (SEQ ID | | | | | |
| NO: 4) | | | | | |
| VNRLSGSRQDLIPS | Q9NQT8 | Kinesin-like protein | 1400 | 0.036914 | 3.722871 |
| YSLGSNKGRWES | | KIF13B | | | |
| QQDVS (SEQ ID | | | | | |
| NO: 5) | | | | | |
| LVAENRRYQRSLP | P19823 | Inter-alpha-trypsin | 60 | 0.003109 | 3.548585 |
| GESEEMMEEVDQ | | inhibitor heavy chain H2 | | | |
| VTLYSY (SEQ ID | | | | | |
| NO: 6) | | | | | |
| GVTSLTAAAAFKP | Q96HC4 | PDZ and LIM domain | 354 | 0.013598 | 0.210835 |
| VGSTGVIKSPSWQ | | protein 5 | | | |
| RPNQG (SEQ ID | | | | | |
| NO: 7) | | | | | |
| MPESLDSPTSGRP | Q96HC4 | PDZ and LIM domain | 341 | 0.027831 | 0.177388 |
| GVTSLTAAAAFKP | | protein 5 | | | |
| VGSTG (SEQ ID | | | | | |
| NO: 8) | | | | | |
| VTSLTAAAAFKPV | Q96HC4 | PDZ and LIM domain | 355 | 0.031096 | 0.172871 |
| GSTGVIKSPSWQR | | protein 5 | | | |
| PNQGV (SEQ ID | | | | | |
| NO: 9) | | | | | |
| SLDSPTSGRPGVTS | Q96HC4 | PDZ and LIM domain | 344 | 0.006705 | 0.128473 |
| LTAAAAFKPVGST | | protein 5 | | | |
| GVIK (SEQ ID NO: | | | | | |
| 10) | | | | | |
| Q8WWL7(1192) | | G2/mitotic-specific | | 0.02828 | 6.854298 |
| | | cyclin-B3 | | | |
| P54886(794) | | Delta-1-pyrroline-5- | | 0.047202 | 5.357437 |
| | | carboxylate synthase; | | | |
| | | Glutamate 5- | | | |
| | | kinase; Gamma-glutamyl | | | |
| | | phosphate reductase | | | |
| P54886(782) | | Delta-1-pyrroline-5- | | 0.047202 | 5.492266 |
| | | carboxylate synthase; | | | |
| | | Glutamate 5- | | | |
| | | kinase; Gamma-glutamyl | | | |
| | | phosphate reductase | | | |

TABLE 10
S-nitrosylation proteins found in ASD subjects

| Name | P-value |
|---|---|
| Polyubiquitin-B | 0.003948 |
| Laminin subunit alpha-1 | 0.003948 |
| Sjoegren syndrome nuclear autoantigen 1 homolog | 0.003948 |
| Glyceraldehyde-3-phosphate dehydrogenase | 0.006485 |
| Pre-mRNA-processing factor 6 | 0.006485 |
| tsc2 | 0.004 |

### Example 2

### Second Cohort Analysis According to the Method of the Invention

The inventors tested a second cohort of 10 subjects afflicted with autism (ASD) and 10 typically developed (TD) male subjects (age-matched: 2-6 yrs.) and built a DFA (ML) model based on the four sets of the multi-omics data (global, phospho-, SNO-proteome from plasma samples and breath volatolome) to distinguish ASD from TD subjects. The algorithm used four features/biomarkers from the 4 omics sets and blind validation determined a significant clustering with high accuracy. The analysis revealed the biomarker signatures as listed under Tables 11-18 below.

TABLE 11
The most significant VOCs found under
the second cohort of ASD subjects

| Substance name | CAS # | P-value |
|---|---|---|
| 2-hydroxy-1-Naphthalenecarboxaldehyde | 708-06-6 | 0.0507 |
| 2,2,4-Trimethyl-1,3-pentanediol | 6846-50-0 | 0.03544617 |
| diisobutyrate | | |
| Hexadecamethyl-Cyclooctasiloxane | 556-68-3 | 0.03197192 |
| methyl ester Hexadecanoic acid | 112-39-0 | 0.00474124 |
| [1,1′:3′,1″-Terphenyl]-2′-ol | 63671-76-1 | 0.09204704 |
| Cyclononasiloxane, octadecamethyl- | 556-71-8 | 0.01243506 |
| Benzene, (1-methyldodecyl)- | 4534-53-6 | 0.08082335 |
| Bis(2-ethylhexyl) phthalate | 117-81-7 | 0.03252503 |
| Dimethyl ether | 115-10-6 | 0.02633223 |
| Squalene | 111-02-4 | 0.03114158 |
| Silanol, trimethyl- | 1066-40-6 | 0.09814397 |
| n-Hexane | 110-54-3 | 0.03273423 |
| Heptanal | 111-71-7 | 0.0083943 |
| Benzaldehyde | 100-52-7 | 0.07358264 |
| Acetophenone | 98-86-2 | 0.01567296 |
| Nonanal | 124-19-6 | 0.00556983 |

TABLE 12
S-nitrosylation proteins found under the second cohort of ASD subjects

| Protein ID | Protein name | P-value |
|---|---|---|
| Q04756 | Hepatocyte growth factor activator; Hepatocyte growth | 2.85E-05 |
| | factor activator short chain; Hepatocyte growth factor | |
| | activator long chain | |
| P00738 | Haptoglobin; Haptoglobin alpha chain; Haptoglobin beta | 0.000237 |
| | chain | |
| P02750 | Leucine-rich alpha-2-glycoprotein | 0.001049 |
| P49815 | Tuberin | 0.001305 |
| P43652 | Afamin | 0.001358 |
| K7ERI9 | Apolipoprotein C-I; Truncated apolipoprotein C-I | 0.001535 |
| P10909 | Clusterin; Clusterin beta chain; Clusterin alpha chain; | 0.001634 |
| | Clusterin | |
| P06727 | Apolipoprotein A-IV | 0.002 |
| Q96PD5 | N-acetylmuramoyl-L-alanine amidase | 0.00204 |
| O75882 | Attractin | 0.002813 |
| P0DP03 | Ig heavy chain V-III region CAM; Ig heavy chain V-III | 0.002925 |
| | region 23 | |
| J3QSE5 | Phosphatidylcholine-sterol acyltransferase | 0.003166 |
| P02675 | Fibrinogen beta chain; Fibrinopeptide B; Fibrinogen beta | 0.004071 |
| | chain | |
| P0DP04 | Ig heavy chain V-III region DOB | 0.005082 |
| K7ER74 | Apolipoprotein C-II; Proapolipoprotein C-II | 0.007724 |
| P04040 | Catalase | 0.00855 |
| P02763 | Alpha-1-acid glycoprotein 1 | 0.009655 |
| A0A075B6K0 | | 0.010582 |
| H0YAC1 | Plasma kallikrein; Plasma kallikrein heavy chain; Plasma | 0.011397 |
| | kallikrein light chain | |
| P49908 | Selenoprotein P | 0.012275 |
| P0D0Y3 | Ig lambda-6 chain C region | 0.014623 |
| Q12805 | EGF-containing fibulin-like extracellular matrix protein 1 | 0.015061 |
| B0YIW2 | Apolipoprotein C-III | 0.016023 |
| P02760 | Protein AMBP; Alpha-1-microglobulin; Inter-alpha- | 0.019801 |
| | trypsin inhibitor light chain; Trypstatin | |
| P01008 | Antithrombin-III | 0.022604 |
| A0A0C4DH38 | | 0.02267 |
| Q5T985 | Inter-alpha-trypsin inhibitor heavy chain H2 | 0.02451 |
| P01011 | Alpha-1-antichymotrypsin; Alpha-1-antichymotrypsin | 0.026052 |
| | His-Pro-less | |
| B4E1Z4 | Complement factor B; Complement factor B Ba | 0.028078 |
| | fragment; Complement factor B Bb fragment | |
| C9JF17 | Apolipoprotein D | 0.028503 |
| P00742 | Coagulation factor X; Factor X light chain; Factor X | 0.029584 |
| | heavy chain; Activated factor Xa heavy chain | |
| P36955 | Pigment epithelium-derived factor | 0.032097 |
| A0A0B4J1V6 | | 0.033197 |
| P06312 | Ig kappa chain V-IV region | 0.036561 |
| P48740 | Mannan-binding lectin serine protease 1; Mannan-binding | 0.038561 |
| | lectin serine protease 1 heavy chain; Mannan-binding | |
| | lectin serine protease 1 light chain | |
| A0A3B3ISJ1 | Vitamin K-dependent protein S | 0.045811 |
| P01834 | Ig kappa chain C region | 0.047914 |
| A0A0C4DH29 | | 0.048079 |

TABLE 13
Proteins having elevated phosphorylation found
under the second cohort of ASD subjects

| Protein | | | Fold |
|---|---|---|---|
| ID | Protein name | P-value | change |
| Q14515 | SPARC-like protein 1 | 1.67E−05 | 29.69 |
| P02671 | Fibrinogen alpha chain; | 0.049555 | 11.89 |
| | Fibrinopeptide A; | | |
| | Fibrinogen alpha chain | | |
| Q9BUN1 | Protein MENT | 0.034323 | 7.84 |
| P49908 | Selenoprotein P | 0.034211 | 6.48 |
| P49908 | Selenoprotein P | 0.024166 | 5.14 |
| P04004 | Vitronectin; Vitronectin V65 | 0.021067 | 4.89 |
| | subunit; Vitronectin V10 | | |
| | subunit; Somatomedin-B | | |
| P19823 | Inter-alpha-trypsin | 0.048825 | 3.67 |
| | inhibitor heavy chain H2 | | |

TABLE 14
Proteins having reduced phosphorylation found
under the second cohort of ASD subjects

| Protein | | | Fold |
|---|---|---|---|
| ID | Protein name | P-value | change |
| P02671 | Fibrinogen alpha chain; | 0.049269 | 0.23 |
| | Fibrinopeptide A; Fibrinogen | | |
| | alpha chain | | |
| Q9Y2W1 | Thyroid hormone receptor- | 0.0252 | 0.22 |
| | associated protein 3 | | |
| P17480 | Nucleolar transcription | 0.015008 | 0.11 |
| | factor 1 | | |
| Q8NBP7 | Proprotein convertase | 0.021862 | 0.09 |
| | subtilisin/kexin type 9 | | |

TABLE 15
Proteins having elevated phosphorylation found under the second
cohort of ASD subjects

| | | | Fold |
|---|---|---|---|
| Protein ID | Protein name | P-value | change |
| Q9H239 | Matrix metalloproteinase-28 | 0.024418 | 6.47 |
| | FFPPLRRLILFKGARYYVLARGGLQVEPYYP | | |
| | (SEQ ID NO: 11) | | |
| Q9H239 | Matrix metalloproteinase-28 | 0.024418 | 6.47 |
| | FPPLRRLILFKGARYYVLARGGLQVEPYYPR | | |
| | (SEQ ID NO: 12) | | |

TABLE 16
Proteins having reduced phosphorylation found under the second cohort
of ASD subjects

| | | | Fold |
|---|---|---|---|
| Protein ID | Protein name | P-value | change |
| Q86US8 | Telomerase-binding protein EST1A | 0.027436 | 0.16 |
| | LAASNPILTAKESLMSLFEETKRKAEQMEKK | | |
| | (SEQ ID NO: 13) | | |
| Q86US8 | Telomerase-binding protein EST1A | 0.027436 | 0.16 |
| | PILTAKESLMSLFEETKRKAEQMEKKQHEEF | | |
| | (SEQ ID NO: 14) | | |

TABLE 17
Proteins having elevated expression levels found
under the second cohort of ASD subjects

| Protein | | | Fold |
|---|---|---|---|
| ID | Protein name | P-value | change |
| P14618 | Pyruvate kinase PKM | 0.021364 | 4.39 |
| Q6UX71 | Plexin domain-containing protein 2 | 2.41E−05 | 4.18 |
| P62805 | Histone H4 | 0.045808 | 4.11 |
| P08246 | Neutrophil elastase | 0.033573 | 3.28 |
| A0A075B6S2 | | 0.00294 | 3.01 |
| P00915 | Carbonic anhydrase 1 | 0.040801 | 2.39 |
| A0A0C4DH25 | | 0.001204 | 2.07 |
| P06310 | Ig kappa chain V-II region RPMI 6410 | 0.016505 | 1.98 |
| P05154 | Plasma serine protease inhibitor | 0.005729 | 1.96 |
| P80108 | Phosphatidylinositol-glycan-specific | 0.022569 | 1.92 |
| | phospholipase D | | |
| P00740 | Coagulation factor IX; Coagulation factor IXa light | 0.023121 | 1.84 |
| | chain; Coagulation factor IXa heavy chain | | |
| P02655 | Apolipoprotein C-II; Proapolipoprotein C-II | 0.002043 | 1.81 |
| P27169 | Serum paraoxonase/arylesterase 1 | 0.007274 | 1.77 |
| P02751 | Fibronectin; Anastellin; Ugl-Y1; Ugl-Y2; Ugl-Y3 | 0.042353 | 1.71 |
| A0A0J9YX35 | | 0.00837 | 1.67 |
| P02749 | Beta-2-glycoprotein 1 | 0.025146 | 1.67 |
| P04180 | Phosphatidylcholine-sterol acyltransferase | 0.000981 | 1.64 |
| P02753 | Retinol-binding protein 4; Plasma retinol-binding | 0.00038 | 1.64 |
| | protein(1-182); Plasma retinol-binding protein(1- | | |
| | 181); Plasma retinol-binding protein(1-179); Plasma | | |
| | retinol-binding protein(1-176) | | |
| P01601 | Ig kappa chain V-I region HK101 | 0.010847 | 1.63 |
| P29622 | Kallistatin | 0.001226 | 1.59 |
| P19823 | Inter-alpha-trypsin inhibitor heavy chain H2 | 0.002783 | 1.54 |
| P02656 | Apolipoprotein C-III | 0.037187 | 1.53 |
| P04278 | Sex hormone-binding globulin | 0.012919 | 1.52 |
| P02766 | Transthyretin | 0.021684 | 1.50 |
| P04196 | Histidine-rich glycoprotein | 0.008096 | 1.49 |
| P05160 | Coagulation factor XIII B chain | 0.013106 | 1.49 |
| P06396 | Gelsolin | 0.004077 | 1.49 |
| P02765 | Alpha-2-HS-glycoprotein; Alpha-2-HS- | 0.029508 | 1.48 |
| | glycoprotein chain A; Alpha-2-HS-glycoprotein | | |
| | chain B | | |
| P19827 | Inter-alpha-trypsin inhibitor heavy chain H1 | 0.004437 | 1.45 |
| P03952 | Plasma kallikrein; Plasma kallikrein heavy | 0.018422 | 1.42 |
| | chain; Plasma kallikrein light chain | | |
| O75882 | Attractin | 0.016077 | 1.40 |
| P49908 | Selenoprotein P | 0.0028 | 1.40 |
| P51884 | Lumican | 0.031099 | 1.40 |
| Q96PD5 | N-acetylmuramoyl-L-alanine amidase | 0.004313 | 1.40 |
| P55058 | Phospholipid transfer protein | 0.017765 | 1.40 |
| O00391 | Sulfhydryl oxidase 1 | 0.049937 | 1.38 |
| P12259 | Coagulation factor V; Coagulation factor V heavy | 0.035854 | 1.35 |
| | chain; Coagulation factor V light chain | | |
| P02786 | Transferrin receptor protein 1; Transferrin receptor | 0.013637 | 1.35 |
| | protein 1, serum form | | |
| P25311 | Zinc-alpha-2-glycoprotein | 0.032556 | 1.34 |
| Q96IY4 | Carboxypeptidase B2 | 0.021375 | 1.32 |
| O75636 | Ficolin-3 | 0.019794 | 1.31 |
| P02787 | Serotransferrin | 0.014128 | 1.30 |
| P10909 | Clusterin; Clusterin beta chain; Clusterin alpha | 0.015028 | 1.27 |
| | chain | | |
| P01042 | Kininogen-1; Kininogen-1 heavy chain; T- | 0.010622 | 1.27 |
| | kinin; Bradykinin; Lysyl-bradykinin; Kininogen-1 | | |
| | light chain; Low molecular weight growth- | | |
| | promoting factor | | |
| P01008 | Antithrombin-III | 0.022118 | 1.24 |
| P01023 | Alpha-2-macroglobulin | 0.018971 | 1.19 |

TABLE 18
Proteins having reduced expression levels found
under the second cohort of ASD subjects

| | | | Fold |
|---|---|---|---|
| Protein ID | Protein name | P-value | change |
| P04003 | C4b-binding protein alpha chain | 0.017437 | 0.82 |
| P01009 | Alpha-1-antitrypsin; Short peptide from AAT | 0.036353 | 0.80 |
| POCOL5 | Complement C4-B; Complement C4 beta chain; | 0.037167 | 0.77 |
| | Complement C4-B alpha chain; C4a | | |
| | anaphylatoxin; C4b-B; C4d-B; Complement C4 | | |
| | gamma chain | | |
| POCOL4 | Complement C4-A; Complement C4 beta chain; | 0.025317 | 0.76 |
| | Complement C4-A alpha chain; C4a | | |
| | anaphylatoxin; C4b-A; C4d-A; Complement C4 | | |
| | gamma chain | | |
| P02763 | Alpha-1-acid glycoprotein 1 | 0.02482 | 0.71 |
| CON_P00761 | | 0.03991 | 0.69 |
| P08571 | Monocyte differentiation antigen | 0.046592 | 0.68 |
| | CD14; Monocyte differentiation antigen CD14, | | |
| | urinary form; Monocyte differentiation antigen | | |
| | CD14, membrane-bound form | | |
| P02748; | Complement component C9; Complement | 0.031813 | 0.67 |
| CON_Q3MHN2; | component C9a; Complement component C9b | | |
| REV_Q4AC99; | | | |
| Q96BY6 | | | |
| P01011 | Alpha-1-antichymotrypsin; Alpha-1- | 0.021538 | 0.66 |
| | antichymotrypsin His-Pro-less | | |
| P02750 | Leucine-rich alpha-2-glycoprotein | 0.001087 | 0.66 |
| P00738 | Haptoglobin; Haptoglobin alpha chain; | 0.024662 | 0.64 |
| | Haptoglobin beta chain | | |
| P02774 | Vitamin D-binding protein | 0.001571 | 0.63 |
| P35542 | Serum amyloid A-4 protein | 0.001712 | 0.55 |
| P18428 | Lipopolysaccharide-binding protein | 0.004382 | 0.43 |
| Q08830 | Fibrinogen-like protein 1 | 0.005616 | 0.21 |
| Q02985 | Complement factor H-related protein 3 | 0.047918 | 0.19 |
| P0DJI8 | Serum amyloid A-1 protein; Amyloid protein | 0.003406 | 0.10 |
| | A; Serum amyloid protein A(2-104); Serum | | |
| | amyloid protein A(3-104); Serum amyloid | | |
| | protein A(2-103); Serum amyloid protein A(2- | | |
| | 102); Serum amyloid protein A(4-101) | | |
| P02741 | C-reactive protein; C-reactive protein(1-205) | 0.002946 | 0.06 |
| P0DJI9 | Serum amyloid A-2 protein | 0.000406 | 0.05 |

Specifically, the inventors showed that the method of diagnosis disclosed herein, utilizing a first model/pattern based on: (i) global expression of Histone H4; (ii) phosphorylation of mitochondrial Rho GTPase 1; (iii) SNO of Tuberin; and (iv) decanal as the VOC, provided diagnosis/prediction accuracy of 92%.

Further, the inventors showed that the method of diagnosis disclosed herein, utilizing a second model/pattern based on: (i) global expression of apolipoprotein C (APOC); (ii) phosphorylation of adenylate cyclase 2; (iii) SNO of apolipoprotein C-1 (APOC1); and (iv) decanal as the VOC, provided diagnosis/prediction accuracy of 90%.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A method of diagnosing an autism spectrum condition in a subject, the method comprising determining in a sample obtained from the subject any one of:
(i) an elevated expression level of at least one biomarker selected from Table 2;
(ii) a reduced expression level of at least one biomarker selected from Table 3;
(iii) phosphorylation of at least one biomarker selected from Table 4;
(iv) S-nitrosylation (SNO) of at least one biomarker selected from Table 5;
(v) a volatile organic compound (VOC) profile comprising at least one VOC selected from any one of Table 1a, Table 1b, Table 1c, Table 1d, Table 1e, and any combination thereof; and
(vi) any combination of (i) to (v),
wherein a significant change of the at least one biomarker in said sample compared to control, is indicative of the subject being afflicted with an autism spectrum condition, thereby diagnosing an autism spectrum condition in the subject.

2.-5. (canceled)

6. The method of claim 1, wherein said VOC profile comprises at least one VOC being detected in a breath sample obtained from said subject, and its corresponding quantity.

7. The method of claim 1, wherein said VOC profile comprises at least one VOC being selected from the group consisting of: phenol, alcohol, esters, ether, ketone, aldehyde, benzene, hydrocarbon, and any combination thereof.

8. The method of claim 1, wherein said VOC profile comprises at least one VOC being selected from the VOCs listed under Table 1a.

9. The method of claim 1, wherein said VOC profile comprises at least one VOC being selected from the VOCs listed under Table 1b.

10. The method of claim 1, wherein said VOC profile comprises at least one VOC being selected from the VOCs listed under Table 1c.

11. The method of claim 1, wherein said VOC profile comprises at least one VOC being selected from the VOCs listed under Table 1d.

12. The method of claim 1, wherein said VOC profile comprises at least one VOC being selected from the VOCs listed under Table 1e.

13. The method of claim 1, wherein said VOC profile comprises a plurality of VOCs selected from the group consisting of the VOCs listed under any one of Table 1a, Table 1b, Table 1c, Table 1d, Table 1e, and any combination thereof.

14. The method of claim 1, wherein said at least one biomarker is selected from Tables 2-5, and wherein said sample is selected from whole blood sample, a serum sample, or a plasma sample.

15. The method of claim 1, further comprising a step of treating said subject determined as being afflicted with an autism spectrum condition with a therapeutically effective amount of therapy suitable for autism.

16. The method of claim 1, comprising determining in a sample obtained from the subject:
(i) an expression level of Histone H4;
(ii) phosphorylation of mitochondrial Rho GTPase 1;
(iii) SNO of Tuberin; and
(iv) a VOC profile comprising decanal,
wherein significant: increase in expression level of Histone H4, phosphorylation of mitochondrial Rho GTPase 1, SNO of Tuberin, and detection of decanal in said VOC profile, in said sample compared to control, is indicative of the subject being afflicted with an autism spectrum condition.

17. The method of claim 1, comprising determining in a sample obtained from the subject:
(i) an expression level of apolipoprotein C;
(ii) phosphorylation of adenylate cyclase 2;
(iii) SNO of apolipoprotein C-1; and
(iv) a VOC profile comprising decanal,
wherein significant: increase in expression level of apolipoprotein C, phosphorylation of adenylate cyclase 2, SNO of apolipoprotein C-1, and detection of decanal in said VOC profile, is indicative of the subject being afflicted with an autism spectrum condition.

18. A kit comprising any one of:
a reagent adapted to specifically determine at least one of:
(i) expression level of at least one biomarker selected from Table 2;
(ii) expression level of at least one biomarker selected from Table 3;
(iii) phosphorylation of at least one biomarker selected from Table 4;
(iv) SNO of at least one biomarker selected from Table 5;
a. a breath collector apparatus for collecting a VOC profile comprising at least one VOC selected from any one of Table 1a, Table 1b, Table 1c, Table 1d, Table 1e, and any combination thereof; and
b. both (a) to and (b).

19. The kit of claim 18, further comprising a control or standard sample.

20. The kit of claim 18, for diagnosing autism spectrum condition in a subject.

21. The method of claim 1, wherein said the diagnosing comprises,
obtaining a sample selected from a breath sample and blood sample from the subject;
obtaining a profile of the sample using an analytic device;
inputting one or more profile into a machine learning model stored in a non-transitory memory and implemented by a processor; and
diagnosing the subject as having or not having an autism spectrum condition based on the output of the machine learning model.

22.-24. (canceled)


SEQUENCE LISTING (14 sequences)

SEQ ID NO: 1 - protein, 31 aa, Artificial Sequence
  Synthetic
EKIFSEDDDYIDIVDSLSVSPTDSDVSAGNI

SEQ ID NO: 2 - protein, 31 aa, Artificial Sequence
  Synthetic
DDYLDLEKIFSEDDDYIDIVDSLSVSPTDSD

SEQ ID NO: 3 - protein, 31 aa, Artificial Sequence
  Synthetic
NAQKQWLKSEDIQRISLLFYNKVLEKEYRAT

SEQ ID NO: 4 - protein, 31 aa, Artificial Sequence
  Synthetic
LSGSRQDLIPSYSLGSNKGRWESQQDVSQTT

SEQ ID NO: 5 - protein, 31 aa, Artificial Sequence
  Synthetic
VNRLSGSRQDLIPSYSLGSNKGRWESQQDVS

SEQ ID NO: 6 - protein, 31 aa, Artificial Sequence
  Synthetic
LVAENRRYQRSLPGESEEMMEEVDQVTLYSY

SEQ ID NO: 7 - protein, 31 aa, Artificial Sequence
  Synthetic
GVTSLTAAAAFKPVGSTGVIKSPSWQRPNQG

SEQ ID NO: 8 - protein, 31 aa, Artificial Sequence
  Synthetic
MPESLDSPTSGRPGVTSLTAAAAFKPVGSTG

SEQ ID NO: 9 - protein, 31 aa, Artificial Sequence
  Synthetic
VTSLTAAAAFKPVGSTGVIKSPSWQRPNQGV

SEQ ID NO: 10 - protein, 31 aa, Artificial Sequence
  Synthetic
SLDSPTSGRPGVTSLTAAAAFKPVGSTGVIK

SEQ ID NO: 11 - protein, 31 aa, Artificial Sequence
  Synthetic
FFPPLRRLILFKGARYYVLARGGLQVEPYYP

SEQ ID NO: 12 - protein, 31 aa, Artificial Sequence
  Synthetic
FPPLRRLILFKGARYYVLARGGLQVEPYYPR

SEQ ID NO: 13 - protein, 31 aa, Artificial Sequence
  Synthetic
LAASNPILTAKESLMSLFEETKRKAEQMEKK

SEQ ID NO: 14 - protein, 31 aa, Artificial Sequence
  Synthetic
PILTAKESLMSLFEETKRKAEQMEKKQHEEF